(19) United States
(12) Patent Application Publication
(10) Pub. No.: US 2004/0138574 A1
(43) Pub. Date: Jul. 15, 2004

(54) Methods and apparatus for enhancing diagnosis of myocardial infarctions

(75) Inventors: Arne Sippens Groenewegen, Burlingame, CA (US); Omar Amirana, San Francisco, CA (US); Terrance Ransbury, Chapel Hill, NC (US); G. Scott Dentino, Capitola, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Resolution Medical, Inc., Chapel Hill, NC (US)

(21) Appl. No.: 10/622,081

(22) Filed: Jul. 16, 2003

Related U.S. Application Data
(60) Provisional application No. 60/396,681, filed on Jul. 17, 2002.

(51) Int. Cl. A61B 5/0402

(52) U.S. Cl. 600/509; 600/515

(57) ABSTRACT

Methods and apparatus for enhancing diagnosis of myocardial infarctions generally compare cardiac data from multiple-lead ECG devices with similar stored data for previous patients with cardiac ischemia. Generally, data from between about 30 and about 130 leads is used to construct body surface maps of multiple patients and those maps are sorted and stored based on presence of ischemia, location of ischemia, size of ischemia and/or the like, for each patient. When a new patient is then evaluated with a multiple-lead ECG, data from that patient can be compared with stored data from the previous patients and the new-patient data can be matched to similar stored data. Thus, characteristics about the new patient's ischemia can be determined, such as presence, location and/or size of a myocardial infarction. The ECG device may be deployed as a set of panels to be applied to the patient, and a display device may be provided to a physician for enhanced diagnosis.

### CROSS-REFERENCE TO RELATED APPLICATION

This is a regular patent application claiming the benefit under 35 U.S.C. §119(e) from U.S. Provisional Patent Application No. 60/396,681 filed Jul. 17, 2002, the full disclosure of which is incorporated herein by reference.

Various embodiments of the present invention employ methods, apparatus, and/or systems related to similar methods, apparatus, and/or systems described in PCT Application No. WO 01/67954 A1, filed Mar. 14, 2001, by a common inventor to the present application, the entire contents of which are hereby incorporated by reference. A copy of WO 01/67954 A1 is attached to this application as Appendix A.

### BACKGROUND OF THE INVENTION

The present invention relates generally to diagnosis of myocardial infarctions. More specifically, the invention relates to methods and apparatus for enhancing diagnosis and localization of acute myocardial infarctions using electrocardiogram data.

Acute myocardial infarction (AMI), commonly known as heart attack, is the single most common cause of death in the U.S. This disease entity is typically caused by an acute thrombotic event which leads to occlusion of a major coronary artery. When a coronary artery is occluded (i.e., blocked), the portion of the heart tissue which that artery supplies with blood no longer receives sufficient blood to provide oxygen to the tissue. If not quickly treated, this lack of blood supply (“ischemia”) results in permanent damage and cell death in the myocardial tissue. Since myocardial damage due to cardiac ischemia becomes irreversible after a certain time, it is imperative that therapy to restore blood flow to the heart be started as quickly as possible. Thus, rapid diagnosis of AMI is also imperative, so that appropriate treatment can be started as quickly as possible.

Typical therapies for AMI include administering thrombolytic agents intravenously or percutaneous transluminal coronary angioplasty (PTCA) with or without stent placement. In either type of therapy, it would be advantageous to have information regarding the location of the AMI on the heart and the identity and/or location of the coronary artery that is blocked. This would allow the medical professional administering the therapy to focus quickly on the affected area for treatment. Information about where on the heart the AMI has occurred and information about the size of the AMI would help a medical professional to determine which artery is occluded and where, along the length of the artery, the occlusion is located.

The most commonly used, currently available methods and apparatus for diagnosing AMI are the standard 12-lead electrocardiogram (ECG) and biochemical cardiac markers, such as creatine kinase isoenzyme-cardiac muscle sub-unit (CK-MB), tropinin T and I, and myoglobin. Less frequently used diagnostic techniques include computerized 12-lead ECG analysis, nuclear imaging (Technetium-99m-sestamibi), echocardiography, ECG exercise stress testing, continuous 12-lead ECG monitoring, and nonstandard ECG leads (i.e. right precordial chest leads). Despite this large number of option, standard 12-lead ECG and cardiac marker analysis remain the current mainstays of AMI diagnosis in the emergency room, likely due to availability and ease of use.

The standard 12-lead ECG and cardiac markers each have several shortcomings in their abilities to quickly and accurately diagnose AMI. For example, 12-lead ECG analysis is a relatively fast diagnostic method but generally has low sensitivity and specificity in detecting AMI, particularly in patients with an acute posterior or lateral MI. On the other hand, the most commonly used cardiac markers (i.e. CK-MB and the cardiac troponins) are not released until several hours after the AMI has occurred and, thus, do not provide for rapid diagnosis. Neither the 12-lead ECG nor the cardiac markers typically provide information regarding the location or size of an AMI.

In an effort to enhance diagnosis of AMI, alternative ECG techniques have been proposed. Some techniques, for example, use greater than the standard number of 12 leads. For example, the PRIME ECG™ device, available from Meridian Medical Technologies, Inc. (Columbia, Md.), includes 64 anterior leads and 12 posterior leads disposed in an electrode vest. Such ECG devices cover a larger area on the chest than conventional 12-lead ECGs, which typically improves their ability to accurately detect AMI. As with conventional ECGs, the multiple-lead ECGs provide rapid diagnosis, as opposed to the slower diagnostic techniques involving cardiac markers.

Although multiple-lead ECGs represent an improvement over conventional ECGs, they still have certain drawbacks. Currently available ECG systems, for example, typically detect AMI by comparing the ECG tracings of a patient to a preset baseline tracing. If the patient's tracing differs from the baseline by more than a specified threshold level, then the patient is said to be experiencing an AMI. Unfortunately, this method of diagnosis can at times lack sensitivity, specificity or both. This leads to some patients with AMI being discharged from the hospital without treatment, because their AMIs are not detected, while other patients are treated for AMI, for example with an angioplasty procedure, when they in fact did not have a AMI. Additionally, although multiple-lead ECGs are able to detect AMI accurately in many cases, they do not provide information regarding the location of the AMI on the heart, the size of the AMI, or similar diagnostic information which would help a cardiologist, surgeon or other treating physician to pinpoint and quantify the AMI.

For the foregoing reasons, it would be advantageous to have methods and apparatus for enhancing diagnosis of AMI. More specifically, it would be advantageous to have improved methods and apparatus for detecting AMI, determining a location of an AMI on a heart, determining the size of an AMI, or any combination thereof.

### BRIEF SUMMARY OF THE INVENTION

The present invention generally uses electrocardiogram data to enhance diagnosis of acute myocardial infarction (AMI). Typically, cardiac data from a patient, acquired from multiple-lead ECG devices (for example, between about 30 and about 130 leads), is compared to stored cardiac data from multiple AMI patients. The stored data also includes information such as presence of an AMI, the location of the AMI on the heart, the size of the AMI, and/or the like. The new patient's ECG can be matched with stored cardiac data that is similar to that of the new patient and, thus, the presence, location, size, and/or the like of the new patient's AMI can be determined.

Generally, data is acquired from multiple patients, sorted, and stored, so that subsequent patients may be diagnosed based on the stored data. For example, cardiac data for each patient may include signals from each of multiple ECG leads. The data from the leads are evaluated and reconstructed to create an integral map and a potential map by evaluating a segment, such as the ST segment, of all the signals and plotting them in a grid or similar matrix to create a unique cardiac map for each patient. The data for multiple patients is then sorted, so that patients with similar cardiac data maps are grouped together. The maps are also correlated with locations of ischemia in the patients' hearts, the locations being determined using standard methods such as thallium scans, angiography, and/or other diagnostic tests. When a new patient is evaluated via the multiple-lead ECG, the presence, location and/or size of any AMI in the patient's heart may be diagnosed by comparing the patient's map to the stored data.

In one aspect, a method for compiling data to enhance diagnosis of a myocardial infarction includes measuring cardiac data of multiple patients, using a multiple-lead electrocardiogram, sorting the cardiac data into groups, each group comprising cardiac data from one or more patients who experienced ischemia in a similar location on their hearts, and storing the sorted cardiac data. Optionally, the measuring may include acquiring electrocardiogram data from the patients with the multiple-lead electrocardiogram while the patients are suspected to be experiencing ischemia, confirming that the patients experienced ischemia, and identifying at least one location of the ischemia on the heart of each patient. Alternatively, the measuring may include acquiring electrocardiogram data from the patients with the multiple-lead electrocardiogram while the patients are experiencing temporary ischemia during percutaneous transluminal coronary angioplasty, and identifying at least one location of the temporary ischemia on the heart of each patient. In some embodiments, the identified location of ischemia on the hearts is taken from a group consisting of an anteroseptal location, an inferior location, a posterolateral location.

In another aspect, a method for enhancing diagnosis of a myocardial infarction in a patient includes matching cardiac data acquired from the patient with stored cardiac data, wherein the stored cardiac data includes data from multiple ischemia patients sorted into groups of patients based on locations on the ischemia patients' hearts where ischemia occurred, and identifying at least one location of ischemia on the patient's heart, based on the stored cardiac data that matches the acquired cardiac data. In various embodiments, the method may also include determining whether a myocardial infarction has occurred in a patient, determining a size of the myocardial infarction, and/or otherwise quantifying the myocardial infarction.

In yet another aspect, a method for enhancing diagnosis of a myocardial infarction in a heart of a patient includes acquiring cardiac data from the heart using a multiple-lead electrocardiogram device, matching the cardiac data to stored cardiac data for at least one ischemia patient, wherein the location of ischemia on the ischemia patient's heart is known, and displaying information regarding at least one location of ischemia on the patient's heart, based on the matching stored cardiac data for the ischemia patient.

A system for using stored electrocardiogram data to enhance diagnosis of a myocardial infarction in the heart of a patient may include a data storage module for storing electrocardiogram data of multiple patients, the patients having either experienced a myocardial infarction or temporary cardiac ischemia during percutaneous transluminal coronary angioplasty and the data being sorted according to locations of ischemia on the hearts of the patients, and computer software for enabling comparison of new electrocardiogram data to the stored electrocardiogram data to determine a location of ischemia in the heart of the patient. The system may optionally include a disposable panel including multiple electrocardiogram leads for acquiring cardiac data from a patient. The system may also optionally include a display module for displaying information related to a patient's heart.

An apparatus for enhancing diagnosis of myocardial infarctions may include a database with stored electrocardiogram data from multiple patients with cardiac ischemia in known locations on the heart, the stored electrocardiogram data being sorted into multiple groups based on the known locations. Alternatively, an apparatus may include a disposable panel or similar device containing multiple electrocardiogram leads for measuring cardiac data in a patient. In other embodiments, an apparatus includes a multiple-lead electrocardiogram panel which may be coupled with a database for diagnosing and localizing ischemia and AMI in a patient.

### DETAILED DESCRIPTION OF THE INVENTION

The present invention generally provides for enhanced diagnosis of myocardial infarctions, especially acute myocardial infarctions (AMIs). More particularly, the invention provides methods and apparatus for helping medical professionals or other users to detect an AMI, identify a location of the AMI on the heart, and/or determine the size of the AMI. From such determinations, a physician can make a more accurate assessment of which coronary artery is occluded and where treatment should be directed.

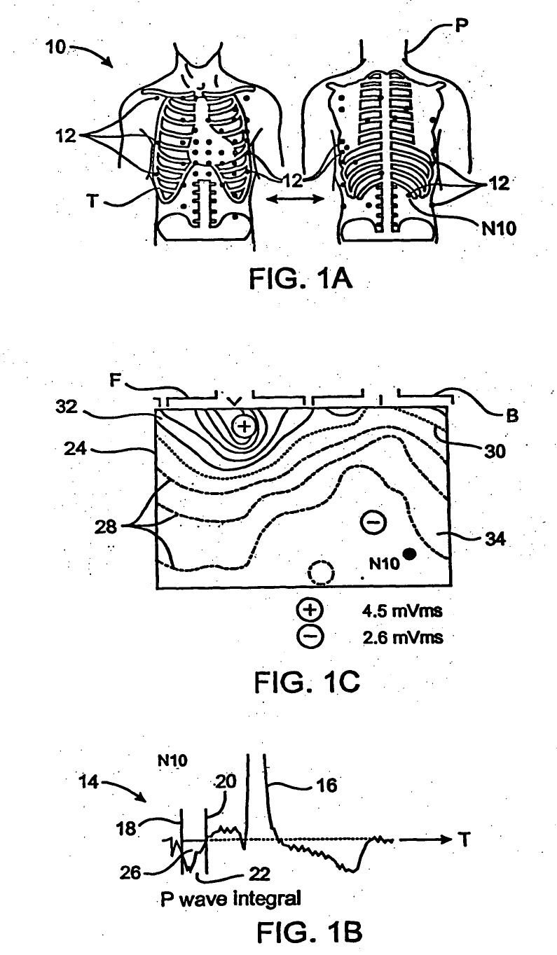

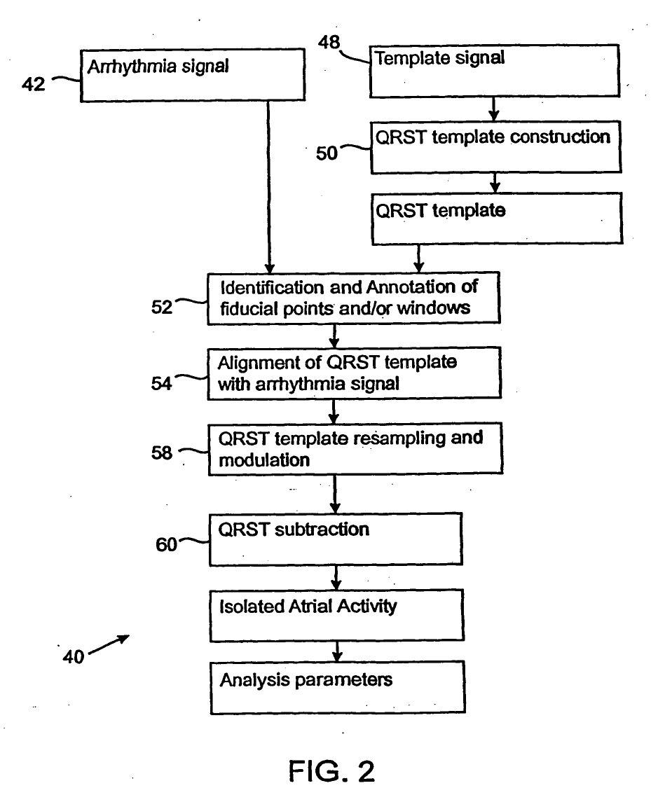

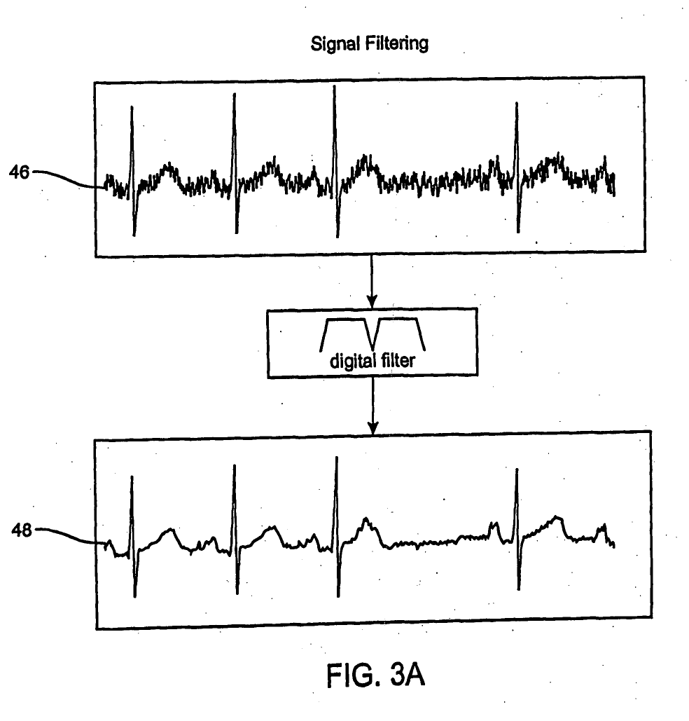

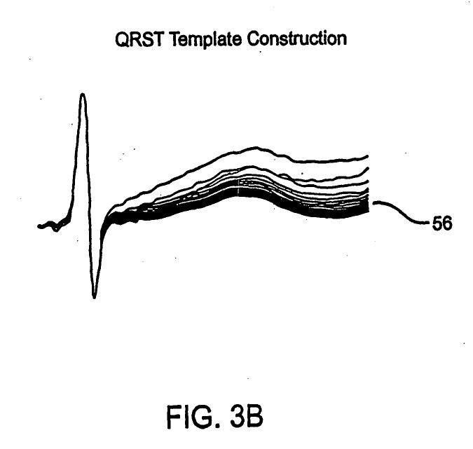

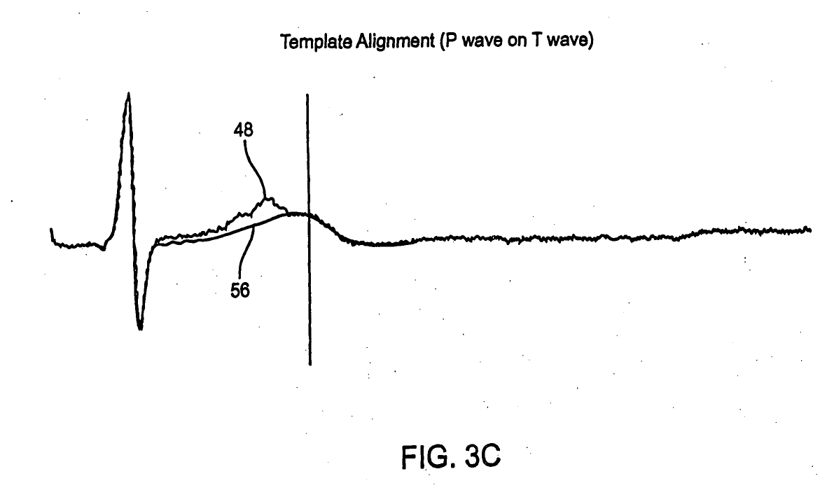

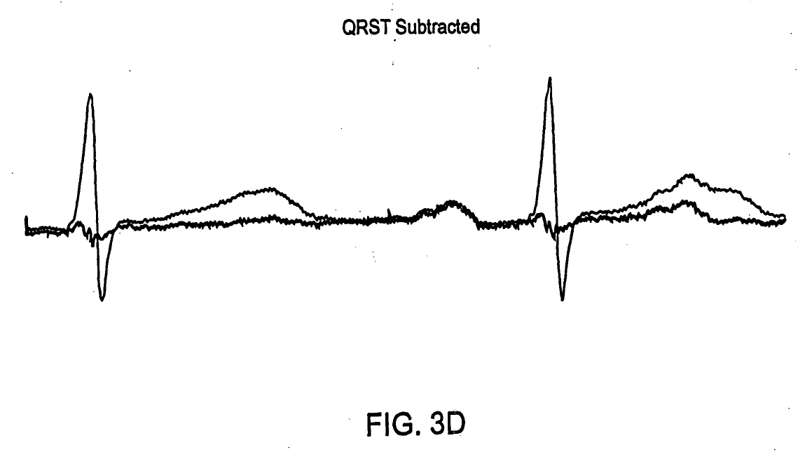

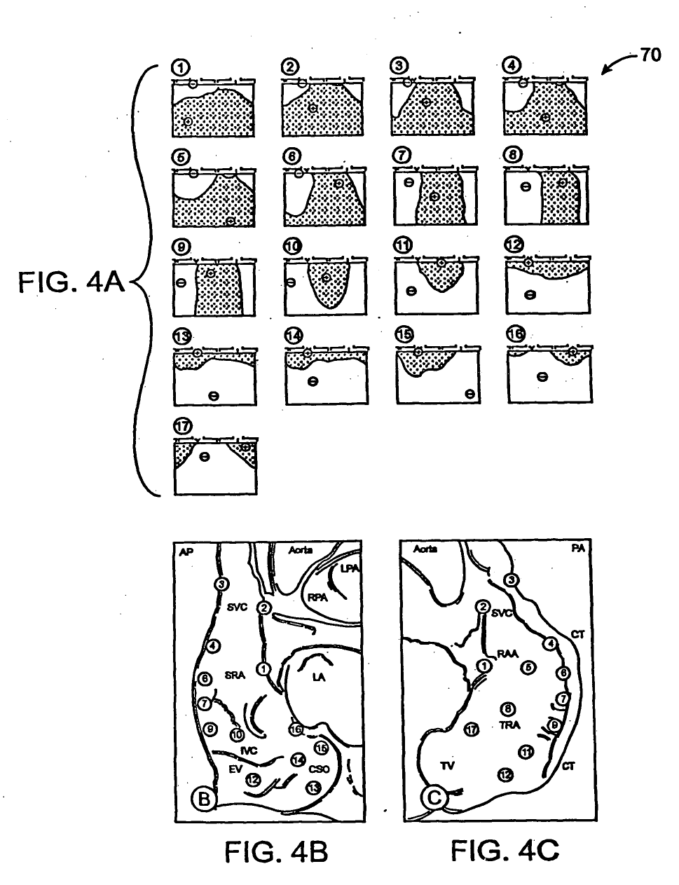

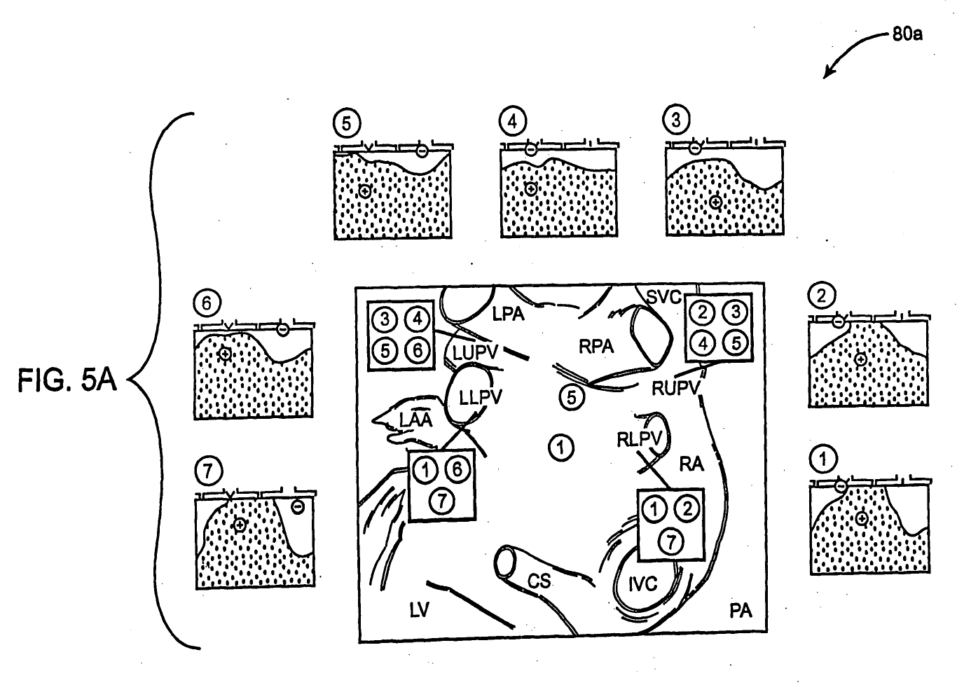

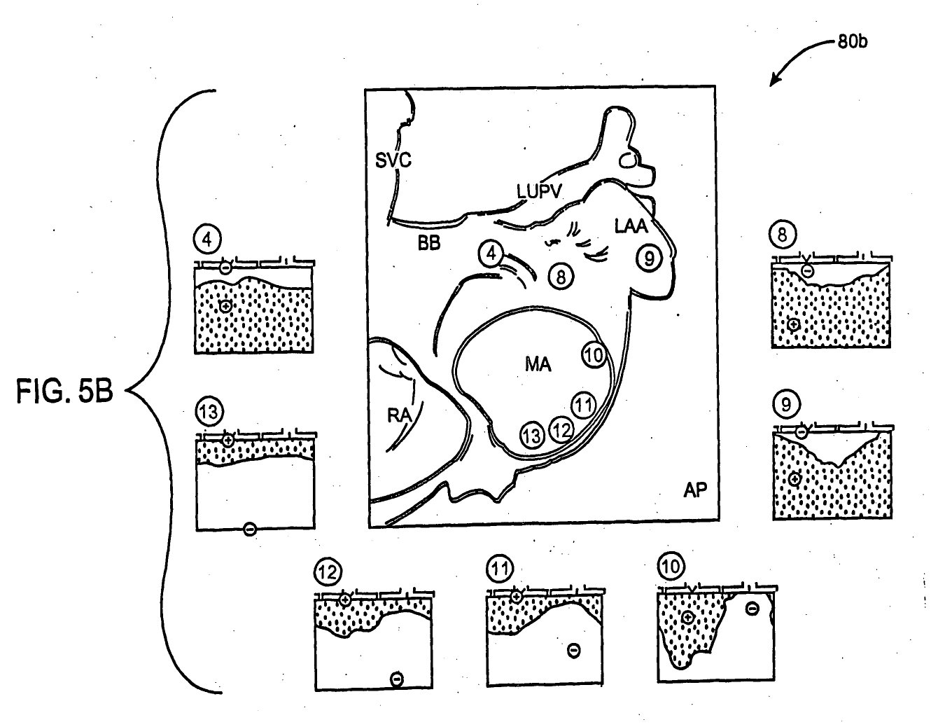

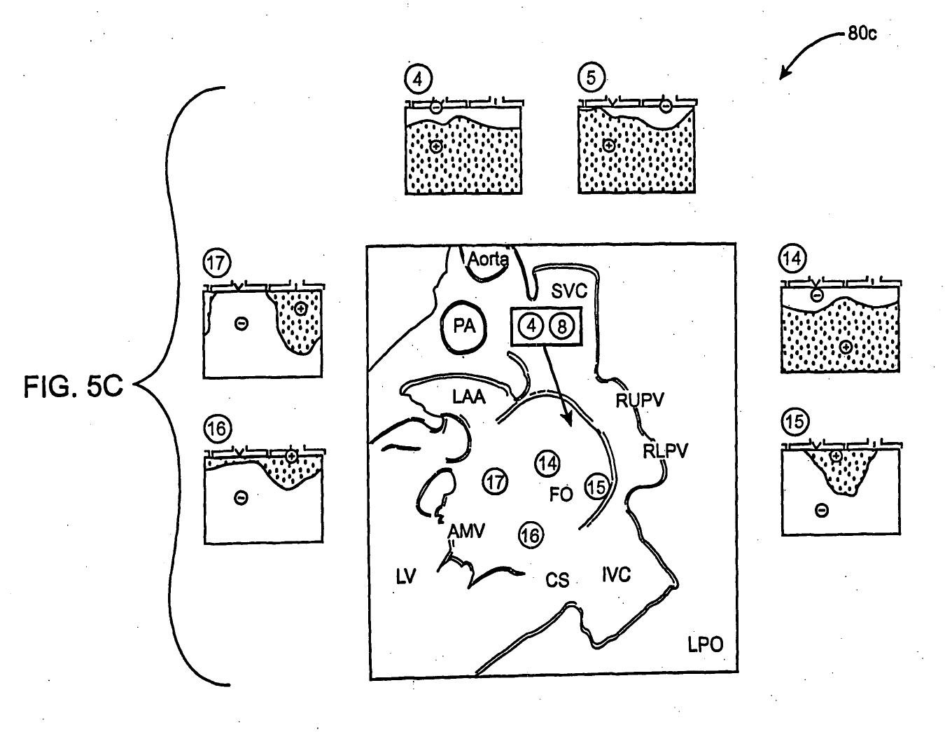

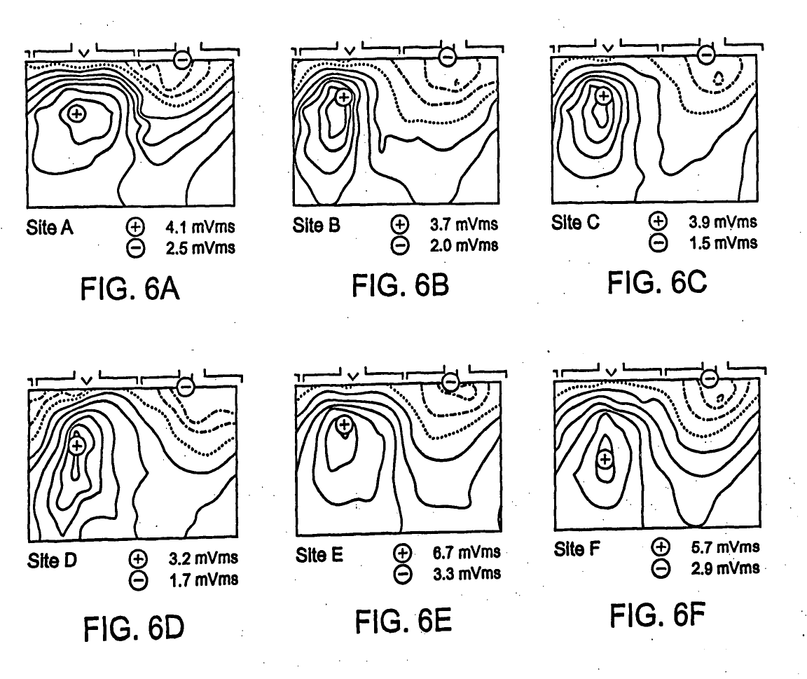

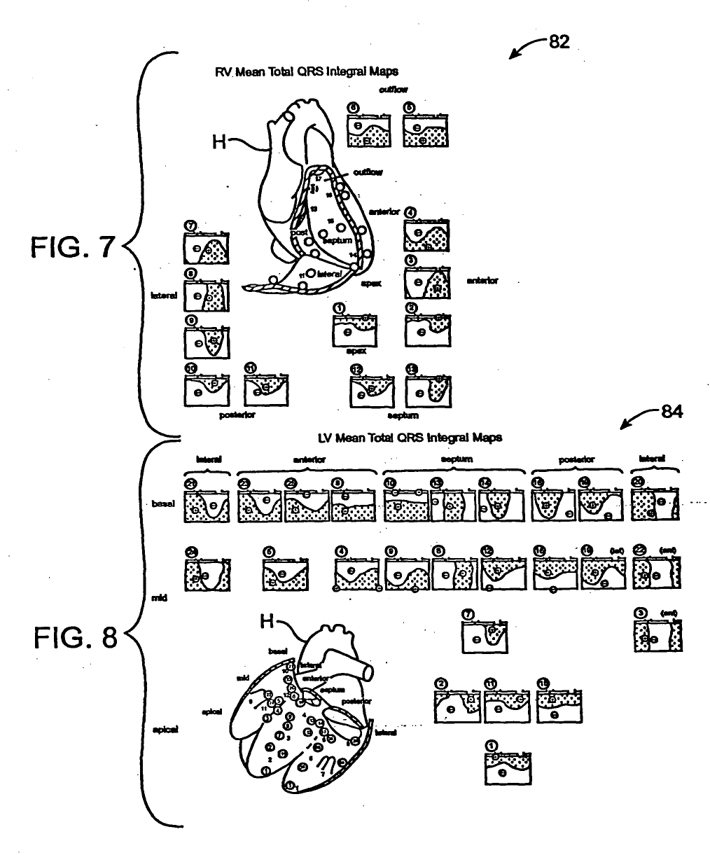

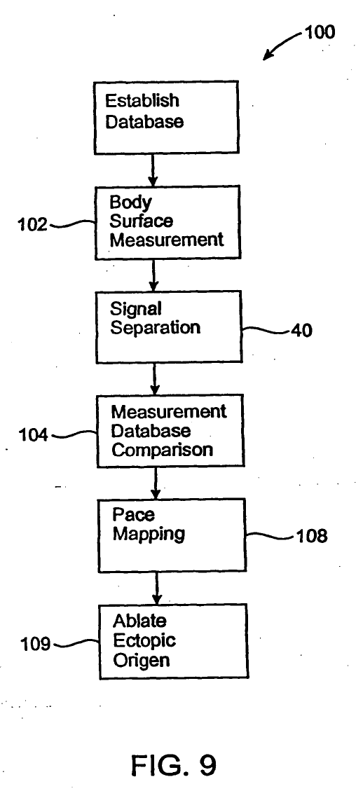

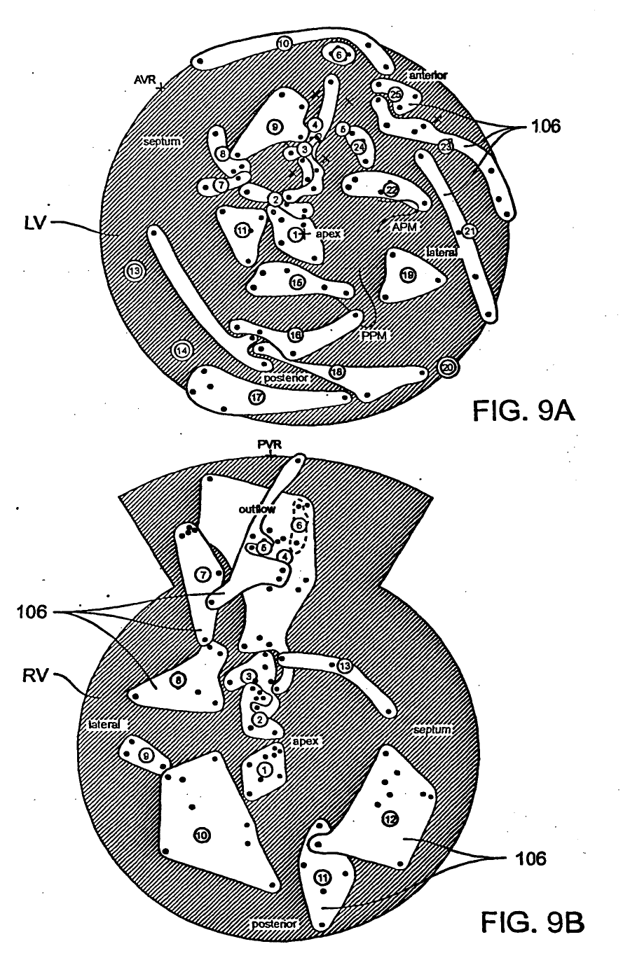

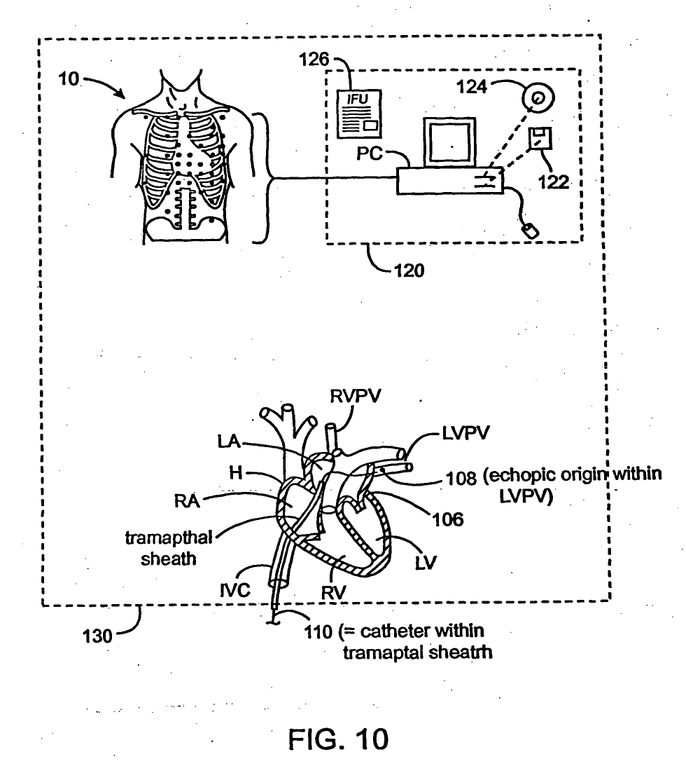

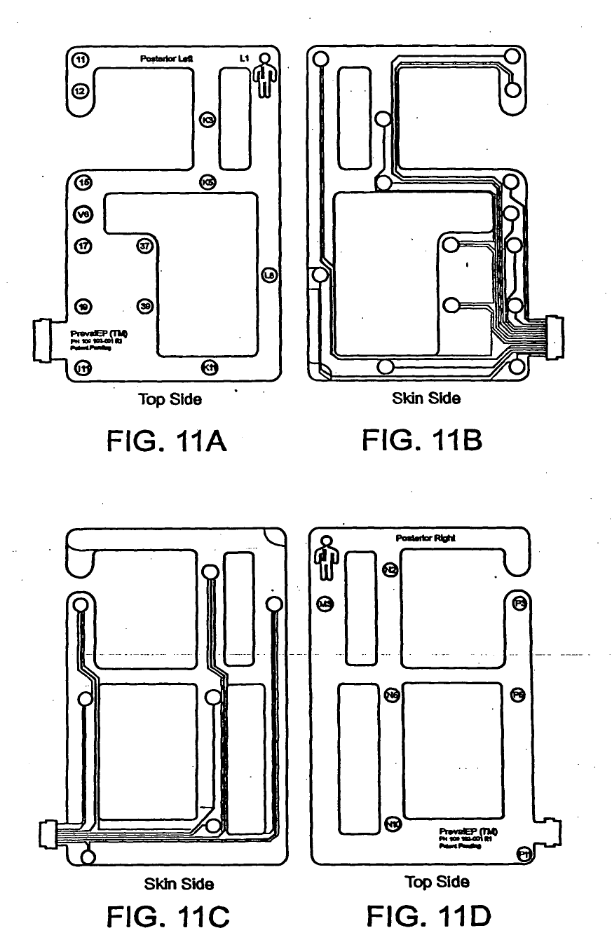

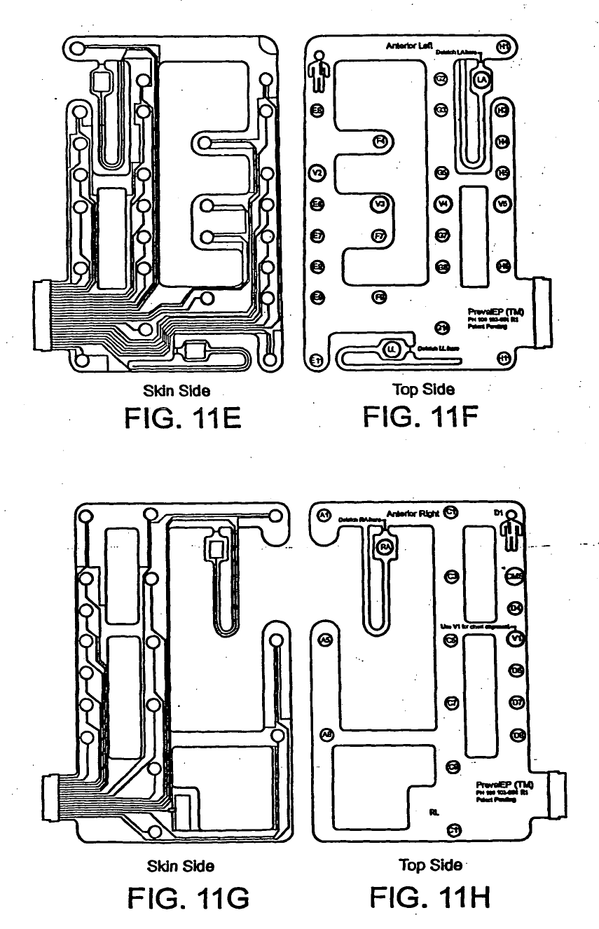

Most embodiments of the present invention employ multiple-lead electrocardiogram (ECG) devices and/or data from such devices to achieve such enhanced diagnosis. In some embodiments, for example, ECGs with between about 30 and about 130 leads are used to compile a body surface map. One type of ECG device which may be used is described in PCT Application No. WO 01/67954 A1, (hereinafter “'954 application”) previously incorporated by reference and attached hereto as Appendix A. Such a device generally includes multiple panels, each panel including multiple ECG leads, which are configured to be placed on, under or around a patient's torso to collect cardiac data. (See, for example, FIGS. 11A-11H and accompanying description in '954 application.) In many embodiments, data from such multiple-lead ECGs are used to create body surface maps, which are compared with stored body surface maps of multiple AMI patients. Again, for descriptions of similar body surface mapping methods and apparatus, in the context of cardiac arrhythmias, see the '954 application. In other embodiments, similar cardiac data is used, but the stored body surface maps are taken from patients who experiences temporary cardiac ischemia while undergoing angioplasty procedures.

Various embodiments of a method for enhancing diagnosis of AMI suitably include first collecting cardiac data. For example, in some embodiments, cardiac data may be acquired from patients undergoing percutaneous transluminal coronary angioplasty (PTCA). Cardiac data may be acquired using any suitable multiple-lead ECG device, but as discussed briefly above, various embodiments employ one or more disposable panels, each of which contains multiple ECG leads. Disposable panels will generally be configured to be placed on, under or around a patient to measure ECG signals with the leads. When a PTCA patient is being treated, he or she will undergo a temporary cardiac ischemia when the angioplasty balloon or similar device is inflated. Cardiac data may be measured during that time, and a map made of that cardiac data may be correlated with the known area of ischemia. The area of ischemia is known, of course, because the surgeon or other physician conducting the PTCA procedure knows where the angioplasty balloon is located when it was inflated.

In other embodiments, patients suspected of having an AMI could be assessed with an ECG device, such as the panels just described. The presence, location and/or size of the AMI could then later be confirmed by cardiac marker analysis, thallium scan, cardiac angiography, and/or other diagnostic methods. Angiography, echocardiography, and/or any other suitable studies may be used to provide further information about the AMI in each patient's heart. For example, the exact location of the AMI on the heart, the occluded coronary artery, the quantity of heart tissue affected by the AMI, and/or the like could be determined. This information could then be stored, along with ECG data, for each patient. In some embodiments, the information regarding the AMI and the ECG data will be processed to determine ECG characteristics that relate to AMI characteristics. For example, a particular ST elevation on an ECG may correspond to a particular location for an AMI in multiple patients. Such correlated information and data may then be used by medical professionals to more specifically diagnose AMI.

In various embodiments, then multilead ECG data will be used to create surface mapping of hearts for use in AMI diagnosis. Typically, such maps will be based on the following three sets of data or characteristics, though some embodiments may include less than all three: (1) infarct detection (rule in/rule out MI); (2) infarct localization (risk assessment to determine nature of acute treatment); and (3) infarct quantification (risk assessment to determine nature of acute treatment). Techniques for mapping are discussed in more detail in the '954 application previously incorporated by reference.

Once maps are made for multiple patients, the maps may be sorted and stored. In many embodiments, for example, maps are generally stored according to the location, on each patient's heart, where ischemia occurred. In very general terms, when a new patient is then diagnosed using a multiple-lead ECG device, data from the new patient can be compared with data from previous patients to determine where the new patient's ischemia is located, whether the patient is actually suffering an AMI, the size of the AMI and/or the like.

For example, in some embodiments criteria for AMI detection will be developed based on the analysis of 62-lead ST integral or potential maps. Such ST maps will typically be developed using the presence of a specific amount of ST elevation (for example >0.1 mV ST elevation) in a required minimum number of chest leads (for example at least 2 leads in the complete array). These data can be obtained by acquiring prospective surface ECG mapping data in a number of patients with AMI documented by cardiac marker analysis and/or coronary angiography. This will enable an assessment of the specific level of ST elevation and the precise minimum number of chest leads required to ensure the highest sensitivity and specificity for AMI detection. In addition, the information provided by the number of leads showing reciprocal ST depression during acute ischemia may also be utilized. As an alternative to the use of ST integral or potential maps, information provided in other segments of the ECG (i.e. QRS complex or QRST segment) may also be used.

In many embodiments, a set of stored data such as database of 62-lead ST integral or potential maps will be developed based on a differentiation of the three major infarct regions/locations of the left ventricle which each relate to the major coronary arteries. These regions are: (1) anteroseptal MI (ASMI) due to an occlusion in the left anterior descending (LAD) coronary artery; (2) inferior MI (IMI) due to an occlusion in the right coronary artery (RCA); and (3) posterior or lateral MI (PMI or LMI) due to an occlusion in the left circumflex coronary artery (LCx). The locations of these infarct regions can be verified using coronary angiography, for example. The classification in IMI may also include the presence or absence of right ventricular infarction (high-risk versus low-risk patient), which again may assessed using coronary angiography. Using methods of various embodiments of the present invention, infarct regions/locations will be specifically related to an area on the chest where a particular ECG change is measured. In some embodiments, for example, the infarct regions will be related to areas on a chest where ST elevation and/or reciprocal ST depression occurs during a specific time instant in a potential map or during a given time interval in an integral map.

As described briefly above, in alternative embodiments, other methods are used to develop the above-described relationships between infarct regions and areas on the chest of ECG change. In some embodiments, for example, 62-lead ECG map patterns may be obtained during percutaneous transluminal coronary angioplasty (PTCA) performed in patients with coronary artery stenosis. In this approach, PTCA is be used as a surrogate model of AMI. At the time when a coronary stenosis is dilated during a PTCA, the vessel occlusion caused by balloon inflation results in temporary transmural ischemia which will generate an ECG pattern similar to the pattern obtained during ischemia caused by AMI. Acquisition of the ECG data during PTCA enables direct correlation of the map patterns with the angiographically validated balloon occlusion site. As an alternative to the use of ST integral or potential maps, information provided in other segments of the ECG (i.e. QRS complex or QRST segment) may also be used.

In some embodiments of the invention, information on the size of myocardial infarcts at each of the three major infarct locations (i.e. LAD, RCA, and LCx) will be obtained by developing criteria for analysis of 62-lead ST integral or potential maps that enable differentiation between patients with a proximal occlusion versus a distal occlusion of the infarct-related artery. In some embodiments, further differentiation may be made, for example by differentiating between proximal, middle, and distal occlusions. The location of a proximal versus a distal occlusion of the infarct-related artery may be verified using coronary angiography. As an alternative, PTCA may again be used as a surrogate model of AMI. Acquisition of the ECG data during PTCA would enable direct correlation of the map patterns with the angiographically validated proximal or distal balloon occlusion site.

In various other embodiments, criteria may be developed for patients with non-ST elevation MI, patients with left (LBBB) or right (RBBB) bundle branch block, left ventricular hypertrophy (LVH), previous (i.e. old) myocardial infarction and/or the like. As with the criteria already discussed, these additional criteria may be determined by examining AMI patients via coronary angiography or by examining PTCA patients. Data from such examinations may then be used to derive criteria related to ECG data.

Once data are collected and one or more sets of criteria for multiple-lead ECG diagnosis of AMI are developed, the data and criteria may be provided to medical professionals in any of a number of suitable forms, to enhance the diagnosis of AMI. In some embodiments, for example, a 62-lead ECG or other ECG device may be coupled with a database such that ECG data collected for a given patient may be quickly compared with data and/or criteria stored in the database. Such comparisons may enable the 62-lead ECG to detect an AMI, determine a location of an AMI, determine a size of an AMI and/or the like.

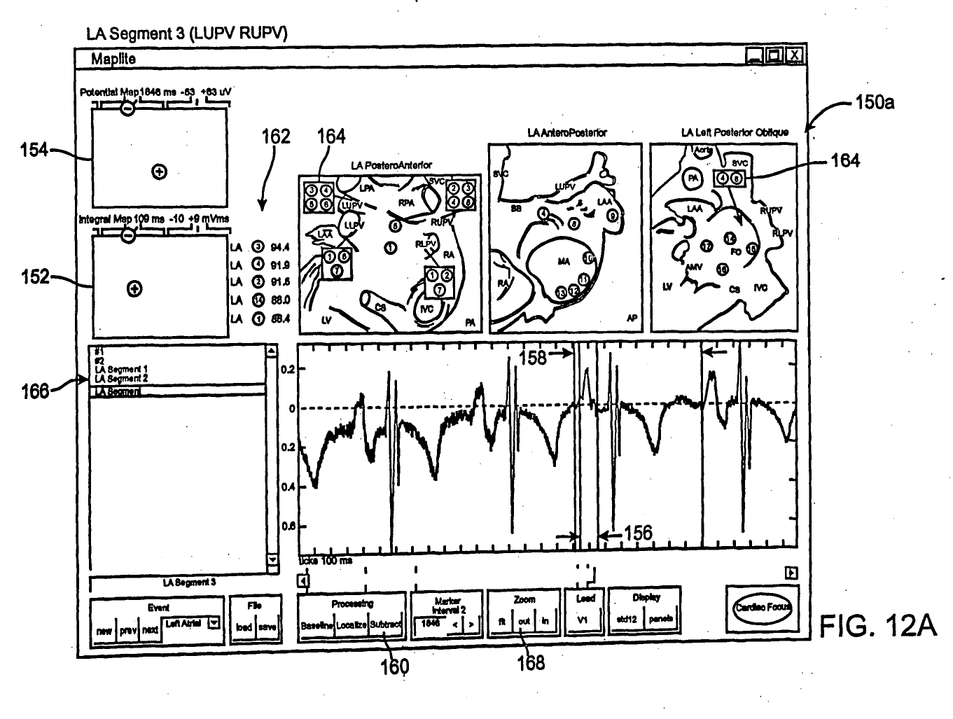

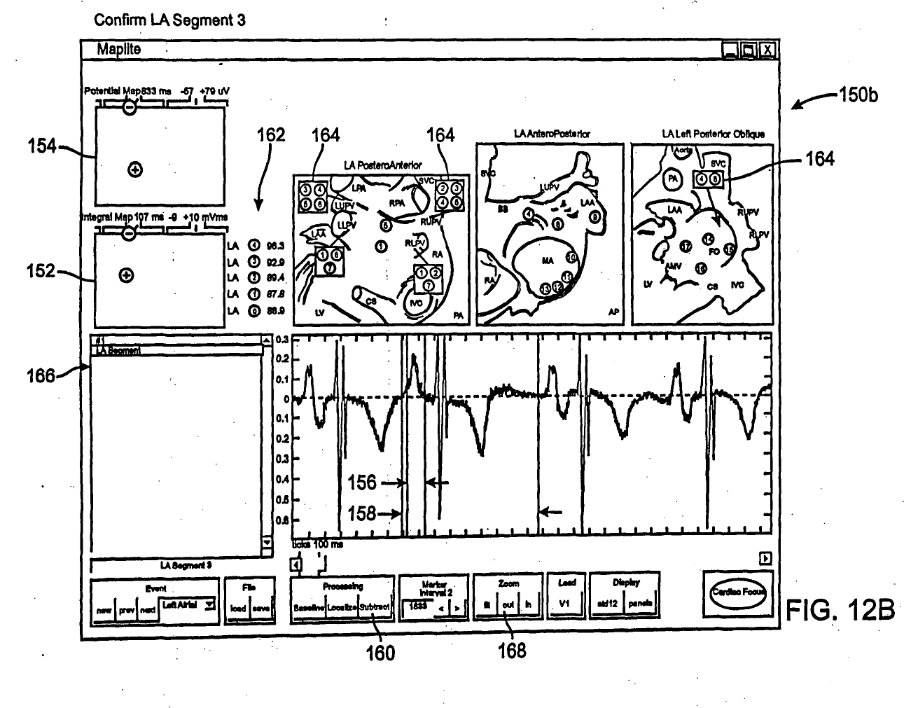

Typically, methods and apparatus of the present invention will provide for display of at least one diagnostic characteristic. For example, in many embodiments the detection of an AMI will be displayed, a location of the AMI will be displayed, and a size of the AMI will be displayed. In other embodiments, an occluded vessel may be listed or displayed and/or a section of the vessel which is occluded may be displayed. Display may be accomplished by any suitable means, such as an LCD screen, a computer screen, and the like. For example, display techniques such as those described in the '954 application may be used. (See, for example, FIGS. 12A-B.) In some embodiments, ECG readings of a patient include intuitive display formats in anatomical models of the heart. Existing imaging techniques may be used to verify the infarct location or infarct-related artery. Such techniques include, but are not limited to, coronary angiography (coronary artery anatomy assessment), nuclear imaging (regional perfusion assessment), and echocardiography (wall motion assessment).

Although the above is a complete description of a number of exemplary embodiments of the invention, other embodiments, as well as variations of the described embodiments, are also contemplated within the scope of the present invention. Therefore, the foregoing description is intended for exemplary purposed only and should not be interpreted to limit the scope of the invention as it is described in the appended claims.

What is claimed is:

1. A method for compiling data to enhance diagnosis of a myocardial infarction, the method comprising:
measuring multiple-lead electrocardiogram data of multiple patients;
sorting the measured data into groups, each group correlating measured data from one or more of the patients with ischemia in a heart region; and
storing the group correlations.

2. The method of claim 1, wherein the measuring step further comprises:
acquiring electrocardiogram data from the patients with the multiple-lead electrocardiogram while the patients are experiencing temporary ischemia during percutaneous transluminal coronary angioplasty; and
identifying at least one location of the temporary ischemia on the heart of each patient.

3. The method of claim 1, wherein the measuring step further comprises:
acquiring electrocardiogram data from the patients with the multiple-lead electrocardiogram while the patients are suspected to be experiencing ischemia;
confirming that the patients experienced ischemia; and
identifying at least one location of the ischemia on the heart of each patient.

4. The method of claim 3, wherein the confirming and mapping steps comprise conducting an angiogram procedure.

5. The method of claim 2 or 3, wherein the at least one location is taken from a group consisting of an anteroseptal location, an inferior location, a posterolateral location.

6. The method of claim 1, wherein the measuring step includes measuring cardiac data using an electrocardiogram device having greater than 12 leads.

7. The method of claim 6, wherein the measuring step includes measuring the cardiac data using an electrocardiogram device having 62 leads.

8. A method for enhancing diagnosis of a myocardial infarction in a patient, the method comprising:
matching cardiac data acquired from the patient with stored cardiac ischemia correlation data, wherein the stored cardiac data is derived from multiple ischemia patients sorted into groups of patients based on locations on the ischemia patients' hearts where ischemia occurred; and
identifying at least one location of ischemia on the patient's heart, based on the stored cardiac data that matches the acquired cardiac data.

9. The method of claim 8, wherein the at least one location is taken from a group consisting of an anteroseptal location, an inferior location, a posterolateral location.

10. The method of claim 8, further comprising determining whether a myocardial infarction occurred in the patient.

11. The method of claim 8, further comprising determining a size of the myocardial infarction.

12. The method of claim 11, wherein determining a size of the myocardial infarction comprises:
defining a relationship between at least one electrocardiogram characteristic and a typical myocardial infarction size; and
comparing the acquired electrocardiogram data and the at least one electrocardiogram characteristic to determine whether the myocardial infarction has the typical myocardial infarction size.

13. A method for enhancing diagnosis of a myocardial infarction in a heart of a patient, the method comprising:
acquiring cardiac data from the heart using a multiple-lead electrocardiogram device;
matching the cardiac data to stored cardiac data derived from at least one ischemia patient, wherein the location of ischemia on the ischemia patient's heart is known; and
displaying information regarding at least one location of ischemia on the patient's heart, based on the matching stored cardiac data for the ischemia patient.

14. A system for using stored electrocardiogram data to enhance diagnosis of a myocardial infarction in the heart of a patient, the system comprising:
a data storage module for storing a correlation database derived from electrocardiogram data of multiple patients, the patients having either experienced a myocardial infarction or temporary cardiac ischemia during percutaneous transluminal coronary angioplasty and the data being sorted according to locations of ischemia on the hearts of the patients; and
computer software for enabling comparison of new electrocardiogram data to the stored electrocardiogram data to determine a location of ischemia in the heart of the patient.

15. The system of claim 14, further comprising:
at least one disposable substrate for placement on the patient, the substrate including multiple electrocardiogram leads; and
at least one display module for displaying information about the patient's heart to a user.

16. The system of claim 17, wherein the display module is configured to provide an integral map of the patient's heart and a three-dimensional image of the patient's heart which shows a location of ischemia.

17. An apparatus for enhancing diagnosis of myocardial infarctions, the apparatus comprising a database derived from electrocardiogram data from multiple patients with cardiac ischemia in known locations on the heart, the database derived by sorting the electrocardiogram data into multiple groups based on the known locations.

18. The apparatus of claim 17, further comprising angiogram or thallium scan data for each of the multiple patients.

19. An apparatus for enhancing diagnosis of myocardial infarctions, the apparatus comprising a disposable panel having multiple electrocardiogram leads, the panel configured for placement on or under a patient to acquire cardiac data from the patient.
